(19) United States
(12) Patent Application Publication
(10) Pub. No.: US 2001/0005714 A1
(43) Pub. Date: Jun. 28, 2001

(54) AMPHIPATHIC MOLECULES AS CHOLESTEROL AND OTHER LIPID UPTAKE INHIBITORS

(76) Inventors: DARIO BOFFELLI, MENLO PARK, CA (US); HELMUT HAUSER, UERIKON (CH)

Correspondence Address: DIKE BRONSTEIN ROBERTS & CUSHMAN, 130 WATER STREET, BOSTON, MA, 021094280

(21) Appl. No.: 09/162,095

(22) Filed: Sep. 28, 1998

Related U.S. Application Data
(63) Continuation of application No. PCT/IB97/00379, filed on Mar. 27, 1997.

(30) Foreign Application Priority Data
Mar. 29, 1996 (GB) 9606686.5
Dec. 24, 1996 (GB) 9626920.4

(51) Int. Cl. A61K 38/00

(52) U.S. Cl. 514/21; 514/2

(57) ABSTRACT

Cholesterol biosynthesis can be inhibited by suitable inhibitors, such as the statins. However, hypercholesterolaemia, whether familial or diet-induced, and more generally hyperlipidaemia are not adequately addressed by cholesterol biosynthesis inhibitors alone, since the body's cholesterol is acquired by uptake from the diet as well as by endogenous synthesis. Lipid is also taken up from the gut. This problem is addressed by providing one or more molecules having amphipathic regions to inhibit the uptake of cholesterol, and other lipids, from the gut. Obesity may also be treated or prevented in this way, as may atherosclerosis. Examples of suitable molecules having amphipathic regions include natural or variant apoproteins and other proteins and peptides having an amphipathic α-helix composed of at least about 15 amino acids.

This invention relates to the use of certain molecules in medicine, particularly as inhibitors of uptake of cholesterol and other dietary lipids from the gut. The invention therefore has application in hyperlipidaemia, including hypercholesterolaemia, and the management of obesity.

Cholesterol is a Janus-faced molecule. On the one hand it is an essential constituent of the plasma membrane of cells, although its precise functional role is still elusive. On the other hand, if too much of it is present and levels of blood cholesterol are high, it is deposited in the wall of arteries, leading to atherosclerotic plaques and eventually to myocardial infarction and stroke. In western industrialised nations, the number of deaths caused by atherosclerosis is greater than by any other disease.

Approximately two thirds of the cholesterol of animal cells are provided by de novo synthesis within cells; the remaining third is of dietary origin and taken up by epithelial cells in the gut. Since cholesterol is insoluble in water and aqueous media such as blood, it has to be dispersed in a stable form. This process is referred to as emulsification and the resulting stable particles are known as serum lipoproteins. The most important transport vehicle of cholesterol in blood is the low-density lipoprotein (LDL) particle (Brown et al., Science 232 34-47 (1986)). The cell's need of cholesterol is taken care of either by the cell's capacity of synthesising cholesterol as mentioned above or alternatively by cells internalising LDL particles from the bloodstream by a mechanism known as receptor-mediated endocytosis. There is an unequivocal causal relation between high levels of LDL in blood and the development of atherosclerosis and in turn myocardial infarction and stroke. The dual role of LDL needs to be stressed: on the one hand LDL particles supply cells with cholesterol, and on the other hand they are responsible for the deposition of cholesterol in the wall of arteries and the development of atherosclerotic plaques.

High blood levels of LDL are either due to a genetic disorder called familial hypercholesterolaemia (FH) or to high-fat diet. The central role of the LDL receptor in hypercholesterolaemia has been emphasised by the work of Brown and Goldstein (Brown et al., Science 232 34-47 (1986). In both cases the number of LDL receptors on the cell surfaces is significantly reduced: in the case of FH the LDL receptors are only partially operative or at worst not functioning at all because of an inherited genetic defect; and in the case of a high-fat, cholesterol-rich diet the synthesis of the LDL receptor is suppressed at the level of transcription. In either case, regardless whether genetic or acquired, the same end resuAlt is produced, namely an, LDL receptor deficiency. As a result, LDL particles are no longer effectively removed from the circulation and its blood level will rise leading to the development of atherosclerosis.

Patients suffering from heterozygous FH have been treated with a class of drugs collectively termed statins, an example of which is simvastatin, marketed by Merck as ZOCOR™. This class of compounds inhibits 3-hydroxy- 3-methylglutaryl coenzyme A (HMG—CoA) reductase, which is the rate limiting enzyme of cholesterol synthesis, thus inhibiting the cells biosynthetic pathway. The statins are often administered in combination with resins, described as bile salt sequestrants, such as cholestyramine. The latter compounds, which are applied orally and in large quantities, were shown to have a lowering effect on the blood cholesterol levels. However, since large quantities have to be used to achieve this effect, and such quantities give rise to undesirable side effects, this class of compounds has not been popular among patients and has not been used widely. Nevertheless Brown and Goldstein showed (Brown et al., Science 232 34-47 (1986)) that the number of LDL receptors can be increased to normal levels in patients with heterozygous FH if these patients are treated with a combination of statins and resins such as cholestyramine.

In 1988, the National Cholesterol Education program in the USA issued a classification of total blood cholesterol and LDL cholesterol levels and recommended dietary therapy for people classified as hypercholesterolaemic. In addition to dietary measures prophylactic approaches leading to the lowering of blood cholesterol would be highly welcome.

The idea of reducing or inhibiting cholesterol absorption in the gut and in turn lowering cholesterol blood levels in this way is old; a great deal of effort has been devoted to this end by the pharmaceutical industry, however, so far with little success. As an example, saponins, administered as a dietary supplement, were shown to reduce blood cholesterol levels in experimental animals and heralded as being potentially useful in the treatment of hypercholesterolaemia (Harwood et al., Journal of Lipid Research 34 377-395 (1993)). However, the approach failed because of difficulties with the supply of saponins. It is practically impossible to obtain pure saponins in large quantities from natural sources. The approach of using synthetic analogues and replacements of saponins has not been successful either, at least not up till now.

In 1990 we reported that absorption of cholesterol by the brush border membrane (“BBM”) of epithelial cells in the gut is protein-mediated (Thurnhofer et al., Biochemistry 29 2142-2148 (1990)). This is also true for esters of cholesterol (Compassi et al., Biochemistry 34 16473-(1995) and other dietary lipids (Thurnhofer et al., Biochim. Biophys. Acta. 1024 , 249-262 (1990)). Our findings are at variance with the widely accepted view documented in text books and review articles that lipid absorption is a passive process involving the diffusion of dietary lipids along a concentration gradient. Our discovery opens new ways and possibilities of interfering and possibly inhibiting cholesterol absorption in the gut. Prior to 1990, approaches taken towards this goal may be classified as unspecific. Examples are the treatments with polymers such as cholestyramine or plant saponins. These compounds are supposed to interact with bile salts in the gut which transport cholesterol and other dietary lipids to the site of absorption and this interaction renders cholesterol and other lipids inaccessible to livid absorption. That large quantities of these reagents are required in this kind of interaction is an indication that the reaction is unspecific. In contrast, with proteins catalysing cholesterol or, more generally, lipid absorption in the BBM, the approach is different and may be classified as specific. Here the aim is to find or design reagents that specifically interact with the protein(s) involved in lipid absorption and thus inhibit lipid absorption.

It has now been found that a family of proteins whose existence is well known and whose function was believed to have been established, but for which no enteral medical use has been proposed, can act as cholesterol or other lipid-uptake inhibitors. The proteins are apoproteins.

It has also been found that the reason that apoproteins appear to be effective in the present invention is because of the presence of amphipathic a helices in their structure and that, therefore, other molecules containing one or more amphpathi.c regions sharing the relevant characteristics (particuiarly dimensions geometry arnd polarity) of a proteinaceous amphipathic α-helix are useful in the invention.

According to a first aspect of the invention, therefore, there is provided the use of a molecule comprising one or more amphipathic regions, particularly amphipathic helices, in the preparation of a medicament for inhibiting the uptake of cholesterol or other lipids from the gut.

According to a second aspect of the invention, there is provided the use of a molecule comprising one or more amphipathic regions, particularly amphipathic helices, in the preparation of a medicament for enteral administration for treating or preventing hyperlipidaemia, especially hypercholesterolaemia, and/or obesity.

The or each amphipathic region shares the relevant characteristics (particularly dimensions, geometry and polarity) of a proteinaceous amphipathic helix composed of at least 13, 14 or 15 amino acid residues, in increasing order of preference.

The invention therefore enables the provision of a method of inhibiting the uptake of cholesterol or other lipids from the gut, the method comprising administering to a patient or subject a molecule comprising one or more amphipathic regions particularly amphipathic helices.

The invention also enables the provision of a method of treating or preventing hyperlipidaemia, especially hyper-cholesterolaemia, and/or obesity, the methodc comprising enterally administering to a patient or subject an effective amount of a molecule comprising one or more amphipathic regions, particularly amphipathic helices.

Particular proteinaceous molecules comprising several amphipathic α-helices are apoproteins.

As mentioned above, low-density lipoprotein (LDL) is the most important transport vehicle of cholesterol in blood. LDL is one of a family of lipoproteins, which are classified according to increasing density: chylomicrons, chylomicron remnants, very low-density lipoproteins (VLDL), intermediate-density lipoproteins (IDL), low density-lipoproteins and high density-lipo-proteins (HDL). Each lipoprotein has its own function; for example, as mentioned above, LDL is important in the transport of cholesterol in the blood, and HDL is believed to be a scavenger of cholesterol from cells and blood vessels, and their roles in those respects are well established. A lipoprotein is a particle consisting of a core of hydrophobic lipids surrounded by a shell of polar lipids and apoproteins (also referred to as apolipoproteins, and sometimes abbreviated to apos). Ten principal apoproteins—A-1, A-2, A-4, B-48, B-100, C-1, C-2, C-3, D and E—have been isolated and characterised; they are synthesised and secreted by the liver and the intestine. Goodman & Gilman, in “The Pharmacological Basis of Therapeutics”, McGraw-Hill, eighth edition, 1992, give the distribution of the apoproteins in the various lipoproteins as follows:

| Lipoprotein Class | Major Apoproteins |
|---|---|
| Chylomicrons | A-1, A-2, A-4, B-48 |
| Chylomicron remnants | B-48, E |
| VLDL | B-100, C, E |
| IDL | B-100, E |
| LDL | B-100 |
| HDL | A-1, A-2 |

It is envisaged that, in principle, any apoprotein may be useful in the invention. Apoproteins A and C (apo A and apo C) have been shown in an in vitro BBM model to be particularly effective. The B apoproteins may be less preferred, in view of their large size and because of their relative lack of solubility in delipidated form.

While the invention has particular application in the treatment or prevention of disease in humans, it may also be applied to other animals (particularly mammals). It is likely that apoproteins from any particular species (including humans) may be the most appropriate for treating animals of that species, but the cross-species use of apoproteins is also within the scope of the invention.

The use of both natural apoproteins (including all allelic variants) and variants of them is within the scope of the invention. Variants include addition, deletion and substitution mutants; mutants may generally be conservative mutants at least from the point of view of cholesterol (and, more, generally, lipid) uptake inhibition, and will generally exhibit significant amino acid homology with the natural sequences. Significant amino acid homology may include homology of at least 40%, 50%, 60%, 70%, 80%, 90%, 95% or even 99%, on a best match basis, in increasing order of preference. Non-interfering amino acid sequences may be added, and non-essential amino acid sequences may be deleted. In short, suitable variants include those proteins whose secondary structure is sufficiently duplicative or imitative of that of a natural apoprotein to be capable of inhibiting the uptake of lipid, particularly cholesterol, from the gut.

Other molecules having one or more amphipathic regions whose characteristics (such as dimensions, geometry and polarity) correspond to that of an amphipathic α-helix of a natural apoprotein may be regarded as variants of apoproteins in the context of the present invention. However, it is more convenient to consider the various non-apoprotein molecules contactaing suitable amphipathic regions by reference to the classes of compounds to which they belong.

One of the most flexible of such classes is that of natural or synthetic peptides and proteins capable of forming an amphipathic helix, or a plurality of amphipathic helices. An amphipathic helix has a hydrophobic face and a hydrophilic face, by virtue of the nature and configuration of the side chains of the amino acids forming the helix. In a Class A amphipathic helix, cationic residues in the hydrophilic face are near the hydrophobic face and anionic residues are remote from the hydrophobic face. In a Class R amphipathic helix, the hydrophilic configuration is inverted, in that anionic residues in the hydrophilic face are near the hydrophobic face and cationic res dues are remote from the hydrophobic face. Natural apoproteins contain Class A amphipathic helixes: Apo A-1 has eight of them. For this reason, compounds comprirsing one or more Class A amphipathic helixes are preferred.

In a right handed α-helix of a peptide or protein, one turn is constituted by 3.6 amino acids. The height per turn is 5.4 Å; so the length of an α-helix consisting of 18 amino acids is 27 Å, and that of a 15 amino acid α-helix is about 22.5 Å. The peptide backbone of this α-helix runs along the surface of a notional (approximately circular sectioned) cylinder of about 5 Å (±0.5 Å) diameter. Taking the outwardly protruding side chains of the amino acid residues into account, the diameter of the cylinder is about 5 to 8 Å. The side chains, which may be polar, charged or non-polar, project approximately perpendicularly to the long axis of the cylinder. About half of the cylindrical surface is covered by charged and polar amino acid residues, and the other half by non-polar residues. As indicated above, an amphipathic α-helix (class A or class R, as the case may be) has opposing polar and non-polar faces oriented parallel to the axis of the cylinder.

Peptides and Proteins which are useful in the invention include those disclosed in EP-A-0162414 and U.S. Pat. No. 4,643,988, the contents of both of which are incorporated herein by reference to the fullest extent permitted by law. Preferred peptides and proteins capable of forming an amphipathic helix contain a sequence:

A₁—B₁—B₂—C₁—D—B₃—B₄—A₂—C₂—B₅—B₆—A₃—C₃—B₇—C₄—A₄—B₈—B₉ (I)

wherein

each of A₁, A₂, A₃ and A₄ independently represents aspartic acid or glutamic acid, or homologues or analogues thereof;

each of B₁, B₂, B₃, B₄, B₅, B₆, B₇, B₈ and B₉ independently represents tryptophan, phenylalanine; alanine, leucine, tyrosine, isoleucine, valine or α-naphthylalanine, or homologues or analogues thereof;

each of C₁, C₂, C₃ and C₄ independently represents lysine or arginine; and

D represents serine, threonine, alanine, glycine or histidine, or homologues or analogues thereof.

Such peptides exhibit a specific arrangement of amino acid residues which results in an idealised amphipathic helix. The specific positioning of negatively-charged positively-charged, and hydrophobic residues is important for the formation of the amphipathic helix, and thus to the intended functioning of the peptide. Analogues having the positive and negative residues reversed from the placement of charged residues occurring in native apolipoproteins show little or no lipid association. In the 18-residue sequence of the above peptides, positively-charged residues (the “C” group of formula I) should be in positions 4, 9, 13 and 15 and negatively-charged residues (the “A” group of formula I) should be at positions 1, 8, 12 and 16. Hydrophobic residues (the “B” group of formula I) should be placed at positions 2, 3, 6, 7, 10, 11, 14, 17 and 18. The residues serine, threonine, alanine, glycine or histidine are preferred at position 5 (“D”). The specific residues chosen to occupy particular functional positions, e.g., positively-charged positions, may be varied without undue adverse effect or the activity or the peptide. For example, the negatively-charged residues aspartic acid and glutamic acid may be interchanged at any position in the sequence in which a negatively-charged residue is called for. Similarly, lysine or arginine may be placed at any of the positively-charged positions. The preferred hydrophobic residues are tryptophan, phenylalanine, alanine, leucine, isoleucine, valine and α-naphthylalanine.

In some preferred peptides, many of the hydrophobic residue positions are occupied by α-naphthylalanine. Particularly preferred embodiments include those in which the sequence is:

Asp-Trp-αNal-Lys-Alα-Phe-αNal-Asp-Lys-αNal-Alα-Glu-Lys-α Nal-Lys-Glu-Alα-Phe (18naA); or

Ac-Asp-Trp-Leu-Lys-Alα-Phe-Tyr-Asp-Lys-Val-Alα -Glu-Lys-Leu-Lys-Glu-Alα-Phe-NH₂ (Ac-18A-NH₂).

This latter peptide is the subject of Venkatachalapathi et al, PROTEINS: Structure, Function, and Genetics 15 349-359 (1993), the contents of which are incorporated by reference to the fullest extent permitted by law. The corresponding unblocked peptide, 18A, is also a preferred compound.

The amino acids used may be naturally occurring forms, or synthetic amino acids which exhibit exceptional desirable qualities may be employed. For example, the synthetic amino acid α-naphthylalanine shows a greater degree of hydrophobicity than any of the naturally occurring amino acids, and is particularly useful in the peptides of the present invention. Similarly, the substituted amino acid dimethyl lysine is more highly positively-charged than unsubstituted lysine, and may he preferred in certain embodiments. Thus, the substitution of useful analogues or homologues or the naturally occurring amino acids required in the suspect peptides is also contemplated. Either D- or L- of amino acids are suitable for use in the present invention. One potential advantage of D-amino acids is the reduced tendency to enzymic hydrolysis in the gut of peptides and proteins containing them. As foreshadowed above, the C- or N-terminal amino acid may be appropriately blocked or otherwise derivatised in a non-interfering manner; for example the N-terminal amino acid may be acetylated, and the C-terminal amino acid may be amidated. N- and/or C-terminal blocking in this way, as in the preferred peptide Ac-18A-NH₂, may stabilise the α-helix in the presence of lipid.

Although the functional amphicathic helix of the preferred peptides described above consists of a sequence of eighteen amino acids, additions to either end of the eighteen residue peptides may be accomplished without substantially affecting the capacity for helix formation. For example, an extending tripeptide may be added at each end of the basic amphipathic peptide chain to minimise helical end effects. Multiple amphipathic helical domains may also prove useful. Thirty-seven residue peptides which consist of two eighteen residue peptides connected by, for example, proline, also show the ability to form discoidal complexes with phospholipid and to displace native apoproteins from HDL.

However, for the present scheme, the eighteen residue unit appears generally to be important to the formation of a proper helix. Deletion of an amino acidat, for example, the 10th position in the sequence will cause rotation of the polar-nonpolar interface by 100°, and results in a peptide which essentially lacks the capacity to displace native aproteins from HDL. Nonetheless, there are useful and functional molecules in which part of the amphipathic helix (for example residues A₄-B₈-B₉) is deleted. One example is Ac-15A-NH₂, which comprises the fifteen N-terminal amino acids of Ac-18A-NH₂ and those structure is as follows:

Ac-Lys-Alα-Phe-Tyr-Asp-Lys-Val-Alα-Glu-Lys-Leu-Lys-Glu-Alα α-Phe-NH₂ (Ac-15A-NH₂).

Ac-15A-NH₂ has 85% of the cholesteryl oleate uptake inhibition activity of Ac-18A-NH₂, as determined in the brush border membrane vesicle model.

The peptides described above may be synthesised by any number of techniques now available for synthesis of simple and complex low molecular weight proteins. Generally speaking, these techniques involve stepwise synthesis by successive additions of amino acids to produce progressively larger molecules. The amino acids are linked together by condensation between the carboxyl group of one amino acid and the amino group of another amino acid to form a peptide bond. To control these reactions, it is necessary to block the amino group of one acid and the carboxyl group of the other. The blocking groups should be selected for easy removal without adversely affecting the polypeptides, either by racemisation or by hydrolysis of formed peptide bonds. Certain amino acids have additional functional groups, such as the hydroxyl group of tyrosine. It is usually necessary to block these additional groups with an easily removed blocking agent, so that it does not interfere with the desired condensation for the formation of peptide bonds.

A wide variety of procedures exist for the synthesis of polypeptides, and a wide variety of blocking agents have also been devised. Most of these procedures are applicable to the peptides of the present invention. The presently preferred method for synthesis of the subject peptides is the Merrifield technique. In this procedure, an amino acid is bound to a resin particle as an ester bond, and the peptide is generated in a stepwise manner by successive additions of protected amino acids to the growing chain. The general procedure is well known, and has been described in many articles, for example: Merrifield, R. B., Jour. Amer. Chem. Soc. 96 2986-2993, (1964).

However, a modification of the known procedure avoids the usual HF-step for the release of the peptide from the solid support by a transfer hydrogenation procedure with formic acid used as the acid donor instead. This procedure, results in the release of a nearly pure peptide, as well as the removal of protecting groups from the ε-NH₂ groups of lysine, benzyl esters from tyrosine.

Another possibility contemplated by the invention is the linkage of amino acid residues by non-peptide bonds, for example by methods known in the art. This expedient is likely to lead to reduced enzymic hydrolysis in the gut.

More generally, natural apoproteins for use in the invention may be prepared by isolation from natural sources (eg serum) or by other means, such as recombinant DNA technology or peptide synthesis, as discussed above. Apoproteins will preferably, but not necessarily, be isolated to protein homogeneity (in the sense that no other proteins are present in the preparation); further they may, but need not, be isolated to total homogeneity (in the sense that no significant amount of other molecules are present at all). Isolation to protein homogeneity may be the optimum strategy, as some lipid will naturally be associated with the apoprotein in vivo. Indeed, lipidated forms of apo A-1 have been found to be more active than the delipidated molecule and are preferred for that reason. The lipidation may be natural, in which case an apoprotein may be administered as its natural lipoprotein counterpart. However, partially lipidated (or delipidated) apoproteins and alternatively lipidated apoproteins, being associated with a non-natural lipoprotein profile, may also be useful.

Recombinant DNA technology may be used to produce apoproteins in any suitable host. The protein and DNA sequences of some of the apcproteins has been established, as the following representative, but not comprehensive, list shows:

| Rat apo D: | Spreyer et al., EMBO J 9(8) 2479-2484 |
|---|---|
| | (1990); |
| Rat apo A-4: | Boguski. et al., Proc. Nat'l. Acad. Sal. |
| | USA 81(16) 5021-5025 (1984); |
| Rat apo A-1: | Boguski et al., Proc. Nat'l. Acad. Sal. |
| | USA 82 992-996 (1985) |
| Human apo E (ε-4 | Das et al., J. Bid. Chem. 260(10) 6240- |
| allele) | 6247 (1985) |
| Human apo E (ε-2 and | Zannis et al., J. Bid. Chew. 259(9) |
| ε-3 allele): | 5495-5499 (1984); |
| Human apo C-2: | Wei et al., J. Bid. Chem. 260(28) 15211- |
| | 15211 (1985) an (erratum) 261(8) 3910- |
| | (1986); |
| Human apo B-100: | Knott et al., Science 230(4721) 37-43 |
| | (1985) |
| Human apo A-1: | Shoulders et al., Nucleic Acids Res. |
| | 11(9) 2627-2837 (1983); |
| Human apo C-3: | Protter et al., DNA 3(6) 449-456 (1984); |
| Human apo A-4: | Elshourbagy et al., J. Biol. Chem. |
| | 262(17) 7973-7981 (1987); and |
| Human apo A-2: | Knott et al., Nucleic Acids Res. 13(17) |
| | 6387-6398 (1985). |

A fuller list of references may be obtained from the NIH ENTREZ molecular biology database using the query “apo”. Existing sequence information should enable the cloning of the genes (either as cDNAs or genomically) of any as yet uncloned apoproteins by standard methods.

Recombinant apoprotein, other protein or peptide expression may take place in any suitable host, whether microbial (eg bacterial, such as Escherichia coli, or fungal, such as Saccharomyces cerevisiae), insect or mammalian. Depending on the host used, the nature and extent of any post-translational modification (eg glycosylation) may be authentic, different from natural or absent. Any functional apoprotein, whether authentically post-translationally modified or not, is useful in the invention.

One or more different molecules may be administered in the practice of the invention . In fact, if certain natural lipoproteins (including chylomicrons, chylomicron remnants VLD, IDL, LDL and HDL) are administered more than one type of apoprotein will be present; for example, as described above, apo A-1 and apo A-2 may be administered together in HDL and apos A-1, A-2, A-4 and B-48 may be. administered together in chylomicrons.

It will be understood that the invention is not limited to the use of peptides and proteins. Rather, the invention encompasses the use of any molecule having the appropriate dimensions, geometry and polarity, or having a region which does so. Synthetic peptidomimetics or other organic molecules may be useful, as may molecules based on sugars, lipids or other biological entities.

Molecules useful in the invention may be formulated for administration by any convenient route, often in association with a pharmaceutically or veterinarily acceptable carrier. Such a formulation forms a third aspect of the invention.

Formulations for parenteral administration will usually be sterile. Pharmaceutical formulations adapted for parenteral administration include aqueous and non-aqueous sterile injection solutions which may contain anti-oxidants, buffers, bacteriostats and solutes which render the formulation isotonic with the blood of the intended recipient; aqueous and non-aqueous sterile suspensions which may include suspending agents and thickening agents are also within the scope of the invention. The formulations may be presented in unit-dose or multi-dose containers, for example sealed ampoules and vials, and may be stored in a freeze-dried (lyophilised) condition requiring only the additlorn of the sterile liquid carrier., for example water for injections, immediately prior to use. Extemporaneous injection solutions and suspensions mas be prepared from sterile powders, granules and tablets.

However, it is preferred that the molecules useful in the invention be administered enterally, especially orally, since their role in the present invention is to prevent or at least inhibit uptake from the gut.

Oral and other enteral formulations need not be sterile and may be presented in unit- or multi-dose form. Oral formulations may be in the form of solids, such as powders, granules, tablets, capsules (for example hard or soft gelatin capsules) or lozenges, or liquids, such as syrups or elixirs. Fillers and/or carriers may be present as appropriate, and those skilled in the art of pharmaceutical formulation will be able to provide such additional or alternative excipients as may be necessary or desirable; flavouring agents are one example. Any formulation intended for oral administration may be formulated for enteric resistance, so as to assist delivery to the small intestine by avoiding or mitigating digestion of the apoprotein(s) in the stomach or the proximal part of the small intestine. Tablets or capsules may be enteric coated, for example by conventional procedures. Liquid formulations may be effectively rendered enteric resistant by including or being co-administered with a suitable agent such as medium-chain triglycerides.

Enteral compositions other than oral compositions include rectal compositions, which may be in the form of a suppository. Suppositories will generally include a suppository base. such as cocoa butter. Again, partizular formulations containing the active ingredient(s) may routinely be prepared by those skilled in the art of phartraceutical formulation.

The amount of apoprotein or other active molecule to be administered in prophylaxis or therapy will be under the control of the physician or clinician. Routine clinical trials will establish optimum levels. The invention only requires that the amounts administered be effective. By way of guidance, however, in vitro experiments suggest that sufficient apoprotein (measured as apoprotein A-1) should be administered to provide a local concentration in the gut of from 1 to 5 μM; on this basis, from 1 to 10 μM of apo A-1 may be administered, with the optimum probably lying within the range 2 to 5 μM. Other active molecules may be administered within the above range or at other dosages determined to be effective and well tolerated.

The invention is useful in the prevention or treatment of hypercholesterolaemia or other hyperlipidaemia of any origin, whether familial or diet-induced. Oral administration is likely to be preferred for both. The invention therefore provides an orally (or other enterally) administerable treatment for, or prophylaxis of, atherosclerosis.

Since the supply of cholesterol depends on a balance between the biosynthesis of endogenous cholesterol and the uptake, from the gut of exogenous cholesterol; it may be appropriate to co-administer a cholesterol biosynthesis inhibitor. The cholesterol biosynthesis inhibitor may-even be co-formulated with the apoprotein or other molecule usefult in the intention, but that is not essential: it may be administered separately or sequentially, and so it may be independently formulated by any convenient method, including those discussed above. According to a fourth aspect of the invention, there is provided a product comprising a molecule having an amphipathic region, as defined above, and a cholesterol biosynthesis inhibitor for combined, separate or sequential administration in hypercholesterolaemia, or other hyperlipidaemia, prophylaxis or therapy and/or in the prophylaxis or therapy of obesity.

The cholesterol biosynthesis inhibitor may be an HMG—CoA reductase inhibitor. Statins are examples of such compounds. HMG—CoA reductase inhibitors of particular interest include the natural fermentation products compactin and mevinolin (also known as lovastatin), dihydrocompactin, dihydromevinolin, eptastatin, the semi-synthetic analogues of mevinolin disclosed in U.S. Pat. No. 4,293,496, and the compounds disclosed in U.S. Pat. No. 4,444,784, U.S. Pat. No. 4,661,483, U.S. Pat. No. 4,668,699 and U.S. Pat. No. 4,771,071 (including simvastatin) as well as those disclosed in WO-A-9100280 and WO-A-9115482, to take a few examples. One or more cholesterol biosynthesis inhibitors may be used, as appropriate.

Bile acid sequestrants, such as cholestryramine, may be present, or at least additionally administered, if desired. However, such agents will often not be present, since one of the advantages of the invention is that their use can be avoided or at least reduced.

Preferred features of each aspect of the invention are as for each other aspect, mutatis mutandis.

All patent and literature docunments referenced throughout this specification are hereby incorporated by reference to the full extenz allowed by law.

The invention will now be illustrated by the following examples. The examples use various abbreviations, whose meanings are as follows:

| | |
|---|---|
| apo A-1 | apolipoprotein A-1 (or A-I) |
| apo A-2 | apolipoprotein A-2 (or A-II) |
| BBM | brush border membrane |
| BBMVs | brush border membrane vesicles |
| DMPC | dimyristoyl phosphatidyicholine |
| EDTA | ethylendiaminetetraacetic acid disodium |
| | salt |
| FE | familial hypercholesterolaemia |
| HDL | high density lipoprotein |
| LDL | low density lipoprotein |
| PAGE | polyacrylamide gel electrophoresis |
| PC | phosphatidylcholine |
| rpm | revolutions per mm |
| SDS | sodium dodecyl sulphate |
| SUVs | small unilamellar vesicles |
| TCA | trichloroacetic acid |
| Tris | tris [hydroxymethyl] aminomethane |

The examples also refer to the accompanying drawings, in which:

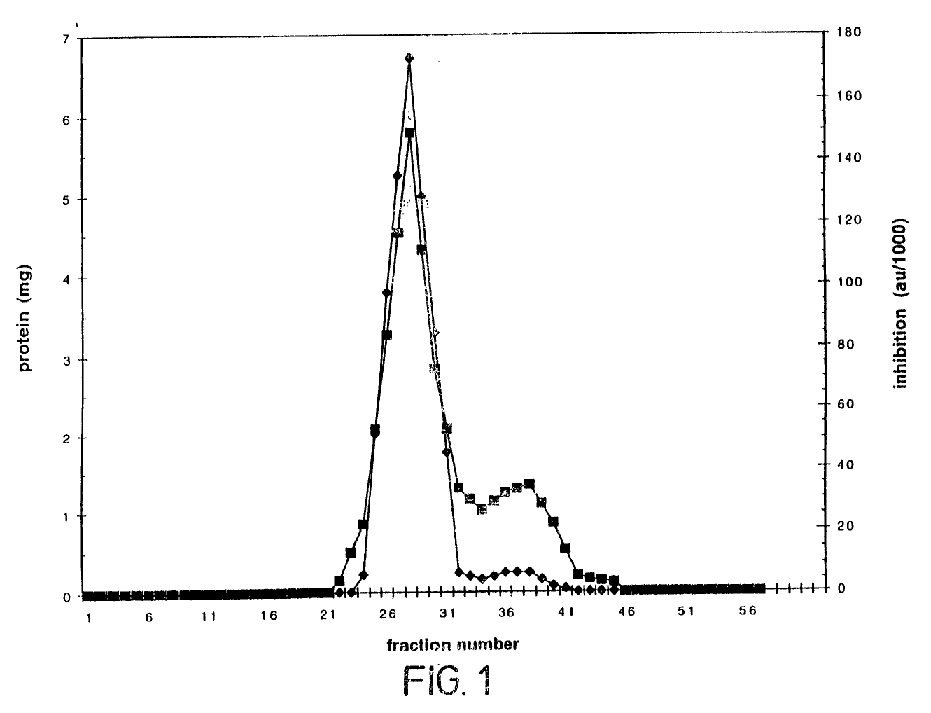

FIG. 1 shows chromatofocussing on PBE94 of partially purified sterol uptake inhibitor protein. The conditions are described in Example 1. The activity peak eluted at fraction 28. Protein concertration was measured with the Pierce BCA^{*} Protein Assay Reagent. Squares represent amount of protein, and diamonds inhibitor activity.

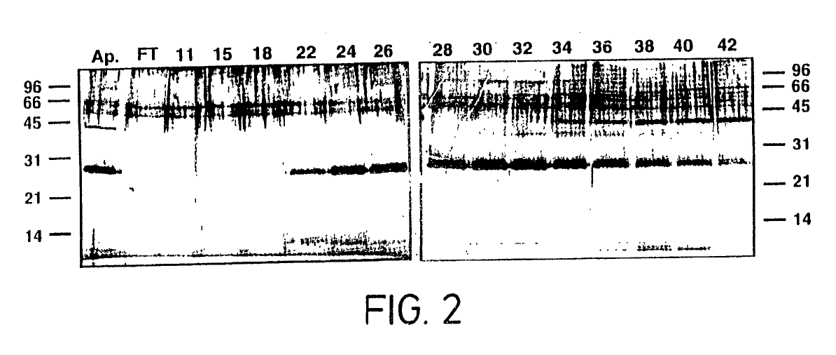

FIG. 2 shows SDS 15% PAGE gels of fractions eluted from the PBE94 column described in Example 1. Electrophoresis was carried out in a Mini-Protean II Dual Slab Cell following the instructions of the manufacturer. The gels were stained with silver. At each side of the gels the electrophoretic mobilities of standard proteins are given together with their molecular masses in kDa. Ap: partially purified sterol uptake inhibitor protein that was applied to the PBE94 column. FT: flow through fraction. 11-42: fractions eluted from the PBE94 column. The double band between 45 and 66 k present in each lane is a silver staining artefact.

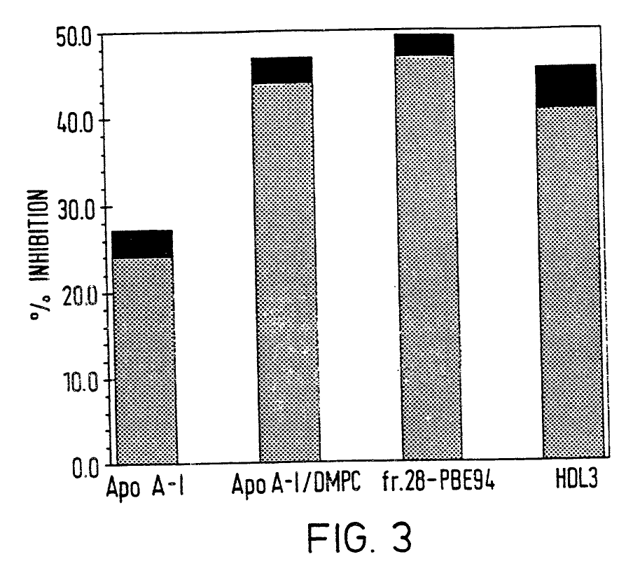

FIG. 3 shows a bar histogram showing the effect of different forms of apoprotein A-1 on cholesterol uptake from egg PC SUVs containing 1 mol % radiolabelled cholesterol as the donor and rabbit BBMV as the acceptors under the conditions described in Example 3. The bars show the percent of inhibition of cholesterol uptake relative to cholesterol uptake in the absence of inhibition. The standard deviation of three different measurements is given by the dark bars on top. Apo A-I:. human apoprotein A-1. Apo A-1/DMPC: human apoprotein A-1 reincorporated into a DMPC bilayer (2.5 mg DMPC/mg apo A-1). Fr.28-PBE94: purified inhibitor eluted in fraction 28 of the PBE94 chromatofocussing column. HDL₃: human high density lipoprotein of density d=1.125-1.21 g/ml.

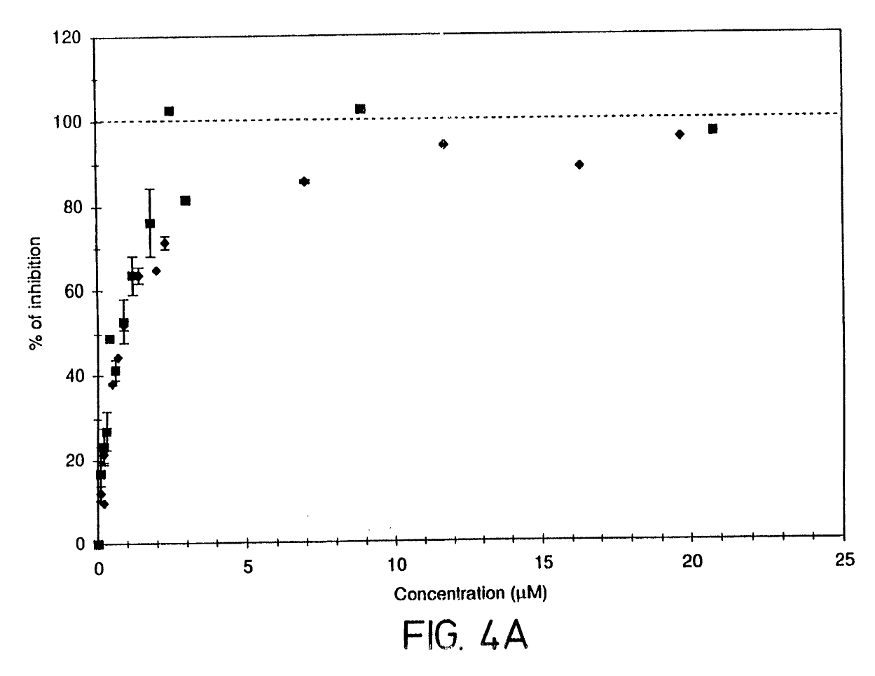

FIG. 4A shows the dose response of cholesteryl oleate uptake, from egg PC SUVs containinq 1 mol % cholesteryl oleate and a trace amount of [³H]-cholesteryl oleyl ether as the donor and rabbit BBMV as the acceptor to increasing amounts of inhibitor protein. Diamonds: inhibition due to human apoprotein A-1. Squares: inhibition due to fr.28-PBE94. Error bars show the standard deviations of three independent measurements.

FIG. 4B shows, in a manner similar to that of FIG. 4A, the inhibitory effect as a function of increasing concentrations of human apo A-1, human apo A-2 and sheep HDL.

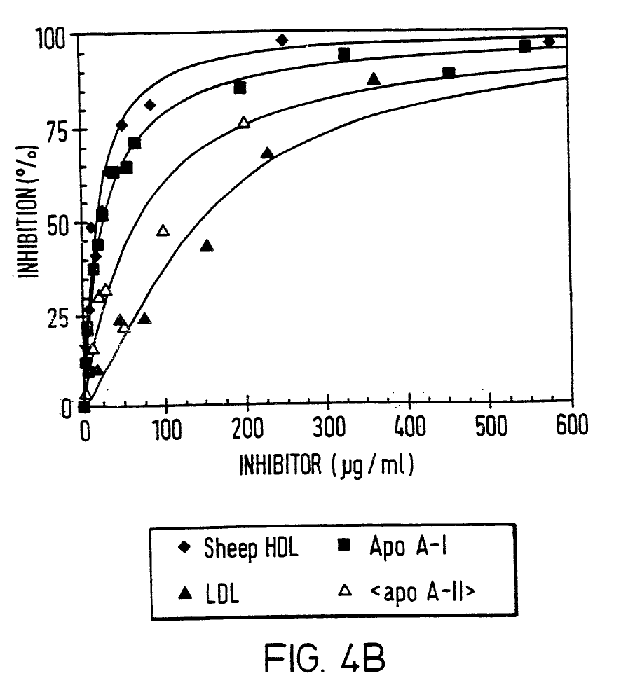

FIG. 5 shows cholesterol uptake by BBMV prepared from normal human duodenum in the absence of inhibitors (●) and in the presence of 60 μM Ac-18A-NH₂ (▪). Phospholipid vesicles at 0.01 mg lipid/ml containing 1 mol % [¹⁴C] cholesterol and BBMV at 0.25 mg lipid/ml were incubated and cholesterol uptake was determined as described in Example 7. Ac-18A-NH₂ was added to the suspension of donor and acceptor vesicles. The data points represent means ± stand. dev. of 3 measurements. The dotted lines represent single-exponential computer fits.

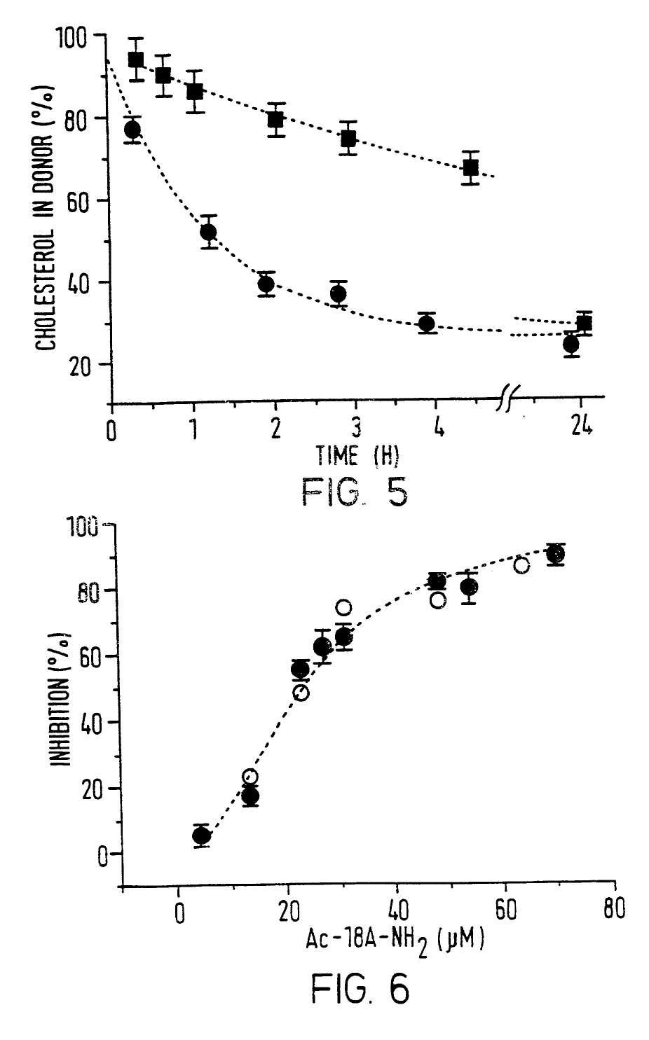

FIG. 6 shows the effect of increasing Ac-18A-NH₂ concentrations on protein-mediated cholesterol uptake by normal (●) and abetalipoproteinemic (◯) BBMV. [¹⁴C] Cholesterol uptake from phospholipid vesicles was determined in the presence of increasing concentrations of Ac-18A-NH₂ using native and proteinase K-treated BBMV. The difference between cholesterol uptake by native and proteinase K-treated BBMV is referred to as protein-mediated cholesterol uptake. The experimental conditions were as described in Example 7; the incubation time was 20 min. the data points for normal BBMV represent means ± stand. dev. of 3 measurements, the dotted line represents the curve fitted to the experimental data according to Rodbard et al, Methods Enzmol. 37 3-22 (1975).

### EXAMPLES

Materials: sodium dextran sulphate, Phenyl SEPHAROSE® 6 Fast Flow (low sub) , SEPHADEX® G-50, PBE™ 94 and POLYBUFFER® 74 were purchased from Pharmacia (Dubendorf, Switzerland), egg PC and dimyristoyl PC from Lipid Products (Nutfield, UK), mouse monoclonal anti-human apolipoprotein A-1 antibodies (unconjugated), and BCA™ Protein Assay Reagent from Pierce (Lausanne, Switzerland), cholesterol (purity ≧99%) and sodium taurocholate (purity ≧97%) from Fluka (Buchs, Switzerland), cholesteryl oleate (purity ≧98%), oleic acid (purity ≈99%) and goat anti-mouse immunoglobulin G (alkaline phosphatase conjugated) from Sigma (Buchs, Switzerland), all radiochemicals used from Amersham (Bucks, UK), Polypropylene ECONO-COLUMNS™ (0.7^{*}4 cm), MINI-PROTEIN® II Dual Slab Cell, Low-Range Molecular Weight Standards and 30% acrylamide/bis solution from BioRad Laboratories (Glattbrugg, Switzerland). All other chemicals were of the best available quality. Water was always double distilled. Frozen sheep serum was obtained from the Basle Institute of Immunology (Basle, Switzerland) and stored at −80° C. prior to use. Human apoprotein A-1 and A-2 and human HDL₃ were a kind gift of Dr. M. C. Phillips of the Medical College of Pennsylvania (Philadelphia, Pa., USA).

### Example 1: The Isolation of a Cholesterol Uptake Inhibitor

The inhibition of sterol uptake activity was measured in an exchange reaction using egg PC SUVs containing 1 mol % [³H]-cholesterol as the donor and rabbit BBMVs as the acceptor. Rabbit BBMvs were prepared according to Hauser et al., Biochimica et Biophysica Acta 602 567-577 (1980)). SUVs of egg PC containing trace quantities of cholesterol and cholesteryl oleyl ether, respectively) were prepared by tip-sonication of the lipid dispersion in Tris/NaCl (50 mM Tris, pH=7.4, 150 mM NaCl, 0.2% NaN₃) as described before (Thurnhofer et al., Biochimica et Biophysica Acta 1024 249-262 (1990)). The donor and acceptor dispersion in Tris/NaCl was centrifuged in a Beckman AIRFUGE™ at 100000 g for 2 min at 4° C. The acceptor dispersion yielded a pellet which was resuspended to a final concentration of 1.7 mg protein/ml with Tris/NaCl and varying amounts of inhibitory activity in the same buffer. This suspension was mixed with an aliquot of the supernatant (top 80%) of the donor dispersion at time zero. The final concentration of the donor in the mixture was 0.2 mg total lipid/ml. The mixture was incubated at 25° C. for 20 min, the exchange reaction was stopped by dilution of the sample with two volumes of Tris/NaCl, and donor and acceptor were separated by centrifugation in the airfuge at 100000 g for 2 min at 4° C. The radioactivities in the supernatant containing donor vesicles and in the pellet containing BBMVs (acceptor) were determined in a Beckman LS 7500 scintillation counter. The results were expressed as percentage of sterol taken up by the acceptor in the presence of the inhibitory activity compared to uptake in the absence of the inhibitory activity.

The inhibitory activity was isolated from sheep serum. Serum was fractionated with dextran sulphate as follows: 100 ml serum were thawed and mixed with 0.5 ml of a 10% sodium dextran sulphate solution in 0.15 M NaCl and 5 ml of 1 M MnCl₂ at room temperature. Unless otherwise noted, all the operations were carried out at room temperature. Precipitation started immediately and was completed by centrifuging the sample at 6000 rpm for 10 min, yielding a supernatant S1 and a pellet P1. S1 was recovered and 6 ml of the 10% dextran sulphate solution and 15 ml of 1 M MnCl₂ were added. The mixture was incubated for 2 hours and then centrifuged at 20000 g for 30 min. The supernatant (S2) was decanted. The pellet (P2) was washed by resuspending with 50 ml Tris/NaCl containing 0.1% dextran sulphate and 0.1 M MnCl₂ and centrifuging as above. The supernatant (S3) was discarded and the pellet (P3) was dispersed with 10 ml of 2% sodium citrate containing 1% NaCl and the pH adjusted to 8 by dropwise addition of 1 M NaOH while stirring. The turbid dispersion was centrifuged at 6000 rpm for 10 min to remove MnO. The supernatant (S4) was recovered. P1 was redissolved with 2 ml of 10% NaHCO₃. MnCO₃ is formed and removed by centrifugation at 500 g for 2 min in a MSE swing-out centrifuge. The supernatant (S5) was recovered and precipitated by adding 100 ml of 50 mM Tris pH 7.4 and 2.5 ml of 2 M MgCl₂ and centrifuging to 6000 rpm for 10 min. The pellet (P6) was resuspended with 2 ml of 5% NaCl and reprecipitated as above two more times. The final pellet (P7) was resuspended with 1.5 ml of 10% sodium citrate and dialysed against Tris pH 7.4 is containing 1% NaCl to remove Mg²⁺. S2, S4 and dialysed P7 were dialysed against 1% BaCl₂, NaCl, centrifuged at 6000 for 10 min to remove precipitated dextran sulphate barium salt and dialysed against Tris/NaCl. Protein concentration ant inhibitory activity were measured and the results are summarised in Table 1.

TABLE 1

| | | | specific activity |
|---|---|---|---|
| | protein | inhibition | (inhibition/mg |
| Sample | (mg) | (au) | protein) |
| serum | 9724 (100) | 4180000 (100) | 430 |
| S2 | 7035 (72) | 370000 (9) | 53 |
| S4 | 667 (7) | 1260000 (30) | 1889 |
| P7 | 39 (0.4) | 138000 (3) | 3540 |
| yield (%) | 80 | 42 | |
| Explanatory note. Dextran sulphate fractionation of sheep serum: sheep serum was sequentially fractionated with dextran sulphate as described above in this example. Protein concentrations were determined with the Pierce BCA Protein Assay Reagent. Inhibitory activity is defined as the percentage of sterol taken up by the acceptor in the presence of inhibitor relative to uptake in the absence of inhibitor and is expressed in arbitrary units (au). | | | |
| # Values in parentheses are percentages of the same quantities relative to sheep serum. | | | |

S4, containing the most of the inhibitory activity, was further purified by hydrophobic interaction chromatography. A column (internal diameter=2.8 cm) was packed with 40 ml of Phenyl Sepharose 6 Fast Flow and equilibrated in 50 mM Tris pH 7.4 containing 2 M NaCl. A flow rate of 4 ml/min was used throughout the chromatographic experiment. Enough solid NaCl was added to S4 (600 mg protein) to reach a concentration of 2 M NaCl. Flow-through proteins were eluted with the same buffer. The column was subsequently washed by lowering NaCl concentration to 0.15 M (fraction 1) and eluted with water (fraction 2) and 15% ethanol fraction 3). Protein concentration and inhibitory activity were measured and the results summarised in Table 2.

TABLE 2

| | | | specific activity |
|---|---|---|---|
| | protein | inhibition | (inhibition/mg |
| fraction | (mg - %) | (au) | protein) |
| S4 | 600 (100) | 11334400 (100) | 1889 |
| flow-through | 362 (59) | 28289 (2) | 78 |
| fraction 1 | 72 (12) | 31148 (2) | 44 |
| fraction 2 | 35 (6) | 955172 (72) | 27290 |
| fraction 3 | 4 (0.6) | 162750 (12) | 40688 |
| yield (%) | 78 | 88 | |

explanatory note. Hydrophobic interaction chromatography of S4: fraction S4 obtained from dextran sulphate fractionation of sheep serum and enriched in sterol uptake inhibitory activity was further purified by hydrophobic interaction chromatography on Phenyl Sepharose S Fast Flow as described above in this example.
Protein concentrations were determined with the Pierce BCA* Protein Assay Reagent. Inhibitory activity is defined as the percentage of sterol taken up by the acceptor in the presence of the inhibitory activity relative to uptake in the absence of the inhibitory activity and is expressed in arbitrary units (au). Values in parentheses are percentages of the same quantities relative to fraction S4.

Fraction 2 was finally purified by chromatofocussing. A column (internal diameter 1 cm) was packed with 20 ml of PBE 94 and equilibrated in 25 mM imidazole-HCl pH 7.3. A flow rate of 0.5 ml/min was used throughout the chromatographic experiment. Fraction 2 (34 mg protein) was applied and the column washed until the absorbance at 280 nm reached the baseline (fraction:PBE-FT). Proteins were eluted with a linear pH gradient generated by POLYBUFFER 74 diluted 1:8 with water and equilibrated at pH=4.0 with HCl. Fractions of 8 ml were collected. Before determination of protein and inhibitory activity of the fractions, Polybuffer 74 was removed by applying 0.5 ml of the fraction to 2.0 ml SEPHADEX G-50 packed in a polypropylene ECONO-Column equilibrated with Tris/NaCl. Recovery of protein and of inhibitory activity was 100% and 82% respectively (FIG. 1). Fractions were analysed by SDS-PAGE as shown in FIG. 2.

### Example 2: Characterisation of the Purified Inhibitor as Apoprotein A-1

The physical characteristics of fraction 28 obtained from the PBE94 column (fr.28-PBE94) as described in Example 1 are summarised in Table 3.

TABLE 3

| Protein | isoelectric point | molecular mass (kDa) |
|---|---|---|
| fr.28-PBE94 | 5.40 | 28.2 (by SDS-PAGE) |
| | | 27.57 (by MS) |
| human apo A-1 | 5.52 | 28.3 |
| rabbit apo A-1 | 5.50 | 25-27 |

explanatory note. Some physical characteristics of the purified inhibitor (fr.28-PBE94): the isoelectric point of fr.28-PBE94 was measured as the pH of elution from the P3E94 column, the molecular mass by either SDS-PAGE or mass spectroscopy (MS). Values for human and rabbit apo A-1 taken from Chapman, Academic Press, Inc. San Diego, New York, Boston, London, Sydney, Tokyo, Toronto 70-143 (1986) are included here for comparison.

Since the characterisation of sheep apo A-1 has not been reported yet, the physical characteristics of human and rabbit apo A-1 are reported for comparison (Chapman, Academic Press, Inc. San Diego, New York, Boston, London, Sydney, Tokyo. Toronto 70-143 1986)). Fr.28-PBE94 was subjected to N-terminal amino acid analysis. The first 29 amino acids of fr.28-PBE94 were 79.3% identical with rat apo A-1, 69.0% with rabbit apo A-1, 86.2% with bovine apo A-1 and 65.0% with human apo A-1. Fr.28-PBE94 cross-reacted with mouse monoclonal anti-human apo A-1 antibodies in Western blotting experiments. Fr.28-PBE94 was shown to contain 0.26 mg total cholesterol (free and esterified) per mg protein, and floated upon centrifugation in a NaBr solution of a density d=1.21 g/ml. This is the floating density of high-density lipoproteins. After subjecting fr.28-PBE94 to the guanidine HCl treatment according to Nichols et al. (Nichols et al., Biochimica et Biophysica Acta 446 226-239 (1976)), fr.28-PBE94 was delipidated, showing the same behaviour as human apo A-1. In order to demonstrate that the inhibitory activity is indeed due to a protein, fr.28-PBE94 and human apo A-1 as a control were either precipitated with 10% trichloroacetic acid or subjected to four cycles of boiling for 5 min and chilling on ice for 5 min. Denatured proteins were removed by centrifugation, and protein and inhibitory activity remaining in the supernatant were determined (Table 4).

TABLE 4

| | protein (%) | inhibition (%) |
|---|---|---|
| human apo A-1 | | |
| after TCA precipitation | 0 | 7.5 ± 1.3 |
| after boiling/chilling | 27 | 18.7 ± 5.1 |
| fr.28-PBE94 | | |
| after TCA precipitation | 0 | −13.1 ± 4.8 |
| after boiling/chilling | 66 | 50.3 ± 3.1 |

explanatory note. Denaturation of the inhibitcry activity: the inhibitory activity was denatured by either exposure to 10% TCA or to 4 boiling/chilling cycles as described above in this example. The results given here are expressed as percent of the protein and sterol uptake inhibitory activity remaining in the supernatant after removal of denatured proteins by centrifugation.

### Example 3: Effect of ano A-1 and apo A-2 on Cholesterol Uptake by Rabbit BBMV

Uptake of sterols (either free or esterified cholesterol) by rabbit BBMVs from SUVs as donors was measured as described in Example 1 in the presence of 20 μg each of human apo A-1, human apo A-1 reincorporated into a DMPC bilayer (2.5 mg DMPC/mg apo A-1), fr.28-PBE94 and human HDL₃: FIG. 3 is a bar histogram showing the inhibition of cholesterol uptake in the presence of: (a) human apo A-1; (b) a lipoprotein complex reconstituted from human apo A-1 and DMPC (1: 2.5=wt ratio) according to Brouillette & Anantharamaiah Biochim. Biophys. Acta 1256 103-109 (1995); (c) sheep apo A-1 purified as described in Example 1 above; and (d) human HDL₃ of a density range d=1.125-1.21 g /ml. The cholesterol absorption was measured at 25° C. using SUV of egg PC containing 1 mol % radiolabelled cholesterol as the donor and rabbit small-intestinal BBMV as the acceptor in the absence and presence of inhibitors. Donor and acceptor both dispersed in 50 mM Tris buffer pH 7.4, 0.15 M NaCl, 0.2% NaN₃ were mixed to final concentrations of 0.05 mg total lipid/ml and 1.7 mg protein/ml, respectively. The amount of apo A-1 in all these samples was kept constant at 20 μg protein/ml. The inhibition in the presence of apo A-1 is expressed as % of the cholesterol uptake measured in the absence of inhibitors. The dark part on top of each bar represents the standard deviation of three measurements.

FIG. 4A shows the inhibition of sterol uptake in the presence of increasing amounts of fr.28-PBE94 (squares) and human apo A-1 (diamonds). Small unilamellar vesicles of egg phosphatidylcholine containing 1 mol % cholesteryl oleate and a trace amount of (1,2-³H₂ (N)]-cholesteryl oleyl ether (37 Ci/mmol, from Amersham, UK) as the donor were made as described in Example 1 and BBMVs as the acceptor were prepared from rabbit small intestine (see Example 1). Donor and acceptor both dispersed in Tris/NaCl buffer (0.05 M Tris HCl pH 7.4, 0.15 M NaCl, 0.02% NaN₃) and a solution of human apo A-1 or sheep HDL in the same buffer were mixed at time zero (total volume: 0.1 ml) so that the final concentrations of donor and acceptor were 0.05 mg/ml total lipid and 1.7 mg protein/ml, respectively. After incubation of the suspension of donor and acceptor in the presence of inhibitor for 20 min. at 25° C., the reaction was stopped by dilution with 2 vol. of Tris/NaCl buffer. Donor and acceptor were separated by centrifugation in the airfuge at 115,000 g for 2 min. at 40° C., and the radioactivities in the supernatant containing donor vesicles and in the pellet containing BBMVs were determined in a BECKMAN™ LS 7500 scintillation counter. Pure human apoA-1 and apoA-2 were prepared from human HDL by delipidation (Scanu & Edelstein, Anal. Biochem. 44 576-588 (1971)) and ion exchange chromatography on Q-Sepharose™ (Weisweiler, Clin. Chim. Acta. 169 249-254 (1987)). The purity of apoA-1 and apoA-II was checked by SDS-PAGE with 8-25% gradient gels using a PHAST™ Electrophoresis System (from Pharmacia). Both proteins gave single bands on overloaded gels. Prior to, use, the proteins were solubilised, in 3 M guanidine HCl and dialysed against the Tris/NaCl buffer.

FIG. 4B shows the inhibitor effect as a function of increasing concentrations of human apo A-1, humfan apo A-2, the sheep HDL and human LDL. Experimental conditions were as described for FIG. 4A. The cholesterol absorption activity of BBMVs measured in the absence of inhibition was taken as 100% and the loss in activity observed in the presence of inhibitor is expressed as %. The experimental points were fitted by the method of Rodbard & Frazier (Methods Enzymol. 37 3-22 (1975)) yielding the solid lines. Sheep HDL (♦), human apo A-1 (▪), human apo A-2 (♦), sheep LDL (▴).

IC₅₀ is the inhibitor concentration at which 50 of inhibition was observed. IC₅₀ values were derived from curve-fittings of the graphs shown in FIG. 4B and are given in Table 5 below:

TABLE 5

| INHIBITOR | IC-50 (μg/ml) |
|---|---|
| Sheep HDL | 17 |
| apo A-I | 25 |
| apo A-II | 66 |
| LDL | 143 |

### Example 4: Effect of Preincubation of Rabbit BBMVs with apo A-1

To demonstrate that the inhibition of sterol absorption is not simply due to apo A-1 (either lipid-free or partially delipidated in the form of fr.28-PBE94) interaction with the donor, rabbit BBMV at 1.7 mg protein/ml were incabated with 0.46 μM human apo A-1 or 0.59 μM fr.28-PBE94 for 5 min. The dispersion was centrifuged in the Beckman airfuge at 100000 g for 2 min at 4° C., the supernatant was removed and the pellet containing rabbit BBMVs and bound inhibitor protein resuspended in an equivalent volume of Tris/NaCl Donor SUVs were added and cholesterol uptake was measured as described in Example 2. Uptake inhibition in the presence of 0.46 μM human apo A-1 or 0.59 μM fr.28-PBE94 was measured as a control. Rabbit BBMVs that were exposed to the inhibitor protein prior to the uptake measurement retained 30±4% of the inhibitory activity measured in the control samples.

### Example 5: Inhibition of Sterol Uptake using Mixed Bile Salt Micelles as Donors

Since bile salt micelles are the most important lipid carriers in the small intestine, it is relevant to measure sterol uptake inhibition using mixed bile salt micelles as the donor. Donor micelles made of 50 mM taurocholate, 6 mM oleic acid and 20 μM radiolabelled cholesterol were prepared as follows: the lipids were mixed at these concentrations in 2:1 chloroform:methanol and the organic solvent was removed by rotary evaporation. The resulting lipid film was dried under high vacuum for at least 1 hour. The dried film was dispersed in the appropriate amount of Tris/NaCl to yield the desired micellar concentration. Acceptor rabbit BBMVs, prepared as described in Example 2, were mixed with either 1.56 μM human apo A-1 or 1.98 μM fr.28-PBE94. Donor mixed micelles were added to the acceptor/inhibitor dispersion to a final concentration of 5 mM taurocholate, 0.6 mM oleic acid and 2 μM radiolabelled cholesterol and the mixture incubated for 10 min at 25° C. The reaction was stopped by centrifuging the mixture in the Beckman airfuge at 100000 g for 2 min at 40° C. Radioactivities in both pellet and supernatant were measured and results evaluated as described in Example 2; incubation with apo A-1 yielded 12% of the inhibitory activity measured at the same inhibitor concentration using SUVs as donor, and incubation with fr.28-PBE94 yielded 23% of the inhibitory activity measured at the same inhibitor concentration using SUVs as donor.

### Example 6: IC₅₀ Values for Various Natural and Variant Apoproteins on Cholesteryl Oleate Uptake at the Brush Border Membrane

The IC₅₀ values for natural (human) apoproteins apo A-I, apo A-II, apo A-III, apo A-IV, apo C-I, apo C-II, apo C-III₁, apo C-III₂ and apo E and variant apoprotein Ac-18A-NH₂ were determined. Ac-18-A-NH₂ is:

Ac-Asp-Trp-Leu-Lys-Alα-Phe-Tyr-Asp-Lys-Val-Alα -Glu-Lys-Leu-Lys-Glu-Alα-Phe-NH₂

and is disclosed in Venkatachalapathi et al, PROTEINS: Structure, Function, and genetics 15 349-259 (1993).

An appropriate amount of inhibitor dissolved in 84.5 μl buffer (0.05 M Tris pH 7.4, 0.15 M NaCl) is mixed in an Eppendorf tube at time zero with 5 μl of a dispersion of donor and 10.5 μl of a dispersion of acceptor (i.e. brush border membrane vesicles (BBMV)) in the same buffer. The final concentration of the donor vesicles was 0.1 mg total lipid/ml, that of the acceptor was 2 mg protein/ml. The resulting mixture was incubated for 20 min at 25° C., and the reaction was stopped by adding 60 μl of the incubation medium to 120 μl ice-cold bufferin an airfuge tube. The diluted dispersion was immediately centrifuiaed in the airfuge at loooooy for 2 min. at 4° C. to separate donor vesicles from BBMV. Two 60 μl aliquots of the donor (=supernatant) were counted in a Beckman LS-7500 liquid scintillating counter to determine the radioactivity remaining in tbe donor.

Preparation of donor vesicles

Small unilamellar egg phosphatidycholine (PC) vesicles containing 1 mol % of cholesterol oleate and a trace amount of ³H-cholesterol oleyl ether were made by dissolving the appropriate amounts of the lipids in CHCl₃/CH₃OH, (2:1, by vol.), taking the solution to dryness on a rotary evaporator and drying the residue in vacuo. The dried lipid film was dispersed in the appropriate volume of buffer by hand-shaking. The lipid dispersion was subjected to tip sonication as described in Brunner et al, J. Biol. Chem. 253 7538-7546 (1978). The resulting donor dispersion in buffer was centrifuged in the airfuge at 100000 g for 2 min at 40° C. Only the top 80% of the donor dispersion after centrifugation was used in the lipid uptake experiment.

Preparation of the BBMV dispersion

BBMV were prepared from frozen rabbit small intestine according to Hauser et al (Biochim. Biophys. Acta 602 567-577 (1980)). Prior to use in the uptake experiment determining IC₅₀ values the BBMV were washed to remove any free protein liberated from the BBM. To this end the BBMV dispersion was diluted with buffer 1:1 in an airfuge tube, and the diluted dispersion was centrifuged in the airfuga at 100000 g for 2 min at 40° C. The supernatant was is carefully decanted, the pellet was resuspended in buffer to the original volume of the BBMV dispersion, and the dispersion was homogenized.

Preparation of apolipoprotein solutions

Solutions of apolipoproteins in buffer were made by dissolving the apolipoprotein in 3 M guanidine HCl to about 1 mg/ml and dialysing the resulting solution exhaustively against the buffer using dialysis tubing with a cutoff of 8 kDa.

IC₅₀ values are shown in Table 6 below.

TABLE 6
IC₅₀ VALUES OF CHOLESTERYL OLEATE UPTAKE AT THE
BRUSH BORDER MEMBRANE

| Apolipoprotein | IC₅₀ (μg/mL) | IC₅₀ (μM) |
|---|---|---|
| Apo A-1 | 14 ± 3 | 0.5 ± 0.1 |
| Apo A-2 | 66 ± 5 | 3.8 ± 0.3 |
| Apo A-4 | 32 ± 4 | 0.7 ± 0.1 |
| Apo C-1 | 12 ± 2 | 1.8 ± 0.4 |
| Apo C-2 | 19 ± 1 | 2.1 ± 0.1 |
| Apo C-3₁ | 4.8 ± 0.8 | 0.6 ± 0.1 |
| Apo C-3₂ | 4.2 ± 0.2 | 0.5 ± 0.2. |
| Apo E | 95 ± 7 | 2.9 ± 0.2 |
| Ac-18A-NH₂ | 35 ± 2 | 16 ± 1 |

Ac-D W L K A F Y D K V A E K L K E A F-NH₂

### Example 7: Further Experiments on Ac-18A-NH₂

Vesicle Preparation

Biopsy samples from human duodenum, 20 to 30 mg of wet tissue each, were suspended in 200 μl buffer (12 mM Tris HCl pH 7.2, 300 mM mannitol, 5 mM EGTA, 1 mM phenylmethylsulfonyl fluoride), frozen in liquid nitrogen and stored at −80° C. prior to use. BBMV were prepared by Mg⁺² precipitation (Hauser et al, Biochim. Biophys. Acta 602 567-577 (1980)) as described in detail by Booth et al (Lancet 1 1066-1069 (1985)). Proteinase K treatment of the BBMV was carried out according to Thurnhofer and Hauser (Biochim. Biophys. Acta 1024 249-262 (1990)). Proteolytic treatment reduced the protein content and specific sucrase activity of the BBMV by - 60%. Small unilamellar phesrholipid vesicles were prepared by tip sonication (Schulthess, Biochemistry 33 4500-4508 (1994)).

Kinetic Experiments

Kinetic experiments were carried out following published procedures (Compassi, Biochemistry 34 16473-16482 (1995) and Tso et al, Am. J. Physiol. 241 G487-497 (1981)).

(I) Egg PC small unilamellar vesicles containing 1 mole (¹⁴C] cholesterol and BBMV both dispersed in 10 mM Tris-HCl pH 7.2, 0.15 M NaCl, 5 mM EDTA were incubated at room temperature. After timed intervals phospholipid vesicles and BBMV were separated by centrifugation at 115000 g for 2 min in a Beckman airfuge. The radioactivities present in pellet and supernatant were determined by counting aliquots in a Beckman LS 7500 liquid scintillatior counter. Fusion of egg PC small unilamellar vesicles and BBMV as a possible mechanism of cholesterol uptake was ruled out as discussed in detail in previous studies (Thurnhofer and Hauser, Biochemistry 29 2142-2148 (1990); Compassi, Biochemistry 34 16473-16482 (1995) and Schulthess, J. Lipid. Res. 37 2405-2419 (1996)).

(II) Similarly, egg PC small unilamellar vesicles containing 1 mol % [¹⁴C] cholesterol as the donor and small unilamellar vesicles of egg PC/egg PA (85:15, mole ratio) as the acceptor were incubated at room temperature. After timed intervals aliquots of the incubation mixture were filtered through DEAE Sepharose C1-CB columns, which retained the negatively charged vesicles. Pure egg PC vesicles were eluted and their radioactivity determined.

The results are shown in FIG. 5.

The experimental data were computer-fitted using the following equation valid for single-exponential exchange reactions: X=X_{∞}+[X_{O}−X_{∞}]e^{-k}1^{[(a+b)/a]t}, where X_{O}, X and X_{∞}represent the fractions of the labelled lipid in the donor at times 0, t and at equilibrium, respectively. K₁ is the pseudo-first-order rate constant of the reaction and a and b are the lipid pools of acceptor and donor, respectively (McKay, Nature 142 997-998 (1938) and Mutsch et al, Biochemistry 25 2134-2140 (1986).

Kinetic measurements were also carried out in the presence of inhibitors; synthetic peptides or apolipoproteins were added to the suppressor of donor and acceptor vesicles. Cholesterol uptake by native and proteinase K treated BBMV was determined in the presence of increasing concentrations of Ac-18A-NH₂, the difference between cholesterol uptake by native and proteinase K treated BBMV is referred as protein-mediated cholesterol uptake in FIG. 6. The IC₅₀ values were determined according to Methods Enzymol. 37 3-22 (1975).

### RESULTS

Cholesterol uptake by human duodenal BBMV was measured in the presence of an amphipathic peptide of composition Ac-Asp-Trp-Leu-Lys-Alα-Phe-Tyr-Asp-Lys-Val-Alα -Glu-Lys-Leu-Lys-Glu-Alα-Phe-NH₂ (Ac-18A-NH₂). This peptide forms an amphipathic α-helix of class A and was shown to mimic some properties of apolipoprotein A-1 (apoA-1) (Methods Enzymol. 128 627-647 (1986)). Acetylation of the H₂N— terminus and amidation of the carboxyl terminus were shown to increase the helicity of the peptide, both in solution and when bound to lipids (Proteins 15 349-359 (1993)). Ac-18A-NH₂ effectively and completely inhibited protein-mediated cholesterol uptake (FIGS. 3 and 4). the concentration of Ac-18A-NH₂ required to reduce protein-mediated cholesterol uptake by 50% (IC₅₀) was determined as 23±1 μM (FIG. 6). Similar inhibition was observed using normal and abetalipoproteinemic BBMV (FIG. 6). In contrast, Ac-18A-NH₂ had no inhibitory effect on passive cholesterol transfer. The residual cholesterol uptake activity by native BBMV which could not be inhibited by Ac-18A-NH₂ at a concentration of 60 μM (squares in FIG. 3) was due to passive cholesterol uptake. It was identical within experimental error with cholesterol uptake by proteinase K-treated BBMV and characterized by a half time of 6.7±1.4 h. Likewise, no inhibitory effect of Ac-18A-NH₂ could be demonstrated for cholesterol uptake by proteinase K-treated BBMV and cholesterol transfer between phospholipid vesicles Table 7).

TABLE A
[14C]Cholesterol uptake by human small-intestinal brush border membrane vesicles and its
inhibition by Ac-18A-NH₂

| Donor | Acceptor | Half time of | Inhibition |
|---|---|---|---|
| (0.01 mg lipid/ml) | (0.25 mg lipid/ml) | cholesterol uptake (*) | IC₅₀ (**) |
| egg PC vesicles | normal human BBMV | 1.0 ± 0.2 h | 23 ± 1 μM |
| egg PC vesicles | abetalipoproteinemlc BBMV | 1.1 ± 0.2 h | 23 ± 3 μM |
| egg PC vesicles | proteinase K-treated normal BBMV | 7.4 ± 1.2 h | no inhibition |
| egg PC vesicles | egg PC/egg PA vesicles | 7.8 ± 1.6 h | no inhibition |

(*) Means ± stand. dev. of 4 experiments (cf. FIG. 1); for abetalipoproteinemic BBMV the error of the experiment is given
(**) Inhibitor concentraticns required to reduce protein-mediated cholesterol uptake by 50%. The error is derived from the fit for the dose response curves (cf. FIG. 6).

That the amphipathic α-helix is the structural principle underlying the inhibition is supported by the observation that the peptide Ac-Asp-Trp-Leu-Alα—Lys-Asp-Tyr-Phe-Lys-Lys-Alα —Leu-Val-Glu-Glu-Phe-Alα—Lys-NH₂ was inactive. This peptide is “scrambled Ac-18A-NH₂” meaning that it has the same amino acid composition as Ac-18A-NH₂ but its amino acid sequence is randomized to eliminate the amphipathic character of the peptide.

The biological relevance of the inhibitory effect observed in vitro is confirmed by an in vivo experiment showing that cholesterol absorption in the small intestine of Sprague-Dawly rats can be inhibited by the amphipathic principle by more than 80%. ApoA-1 added to the diet was used as the inhibitor, because this protein could be purified from human serum in sufficient quantity. Bases or the experimental evidence presented we ppropose that the inhibitory effect on cholesterol uptake is not restricted to particular amphipathic molecules. It is likely that the chemical nature of the amphipathic compound is of secondary importance and that the geometry and the polarity of the compound are the decisive determinants. The results presented here have important implications since amphipathic molecules belonging to any class of biological compounds such as lipids, proteins or carbohydrates might inhibit cholesterol uptake by the brush border membrane.

### EXAMPLE 8

Inhibitory Effect of Amphipathic Helical Peptides of Varying Lengths

The activity (inhibitory effect) of Ac-15A-NH₂, Ac-12A-NH₂ and Ac-9A-NH₂ was determined. The amino acid sequence of these peptides are as follows:

Ac-18A-NH₂: CH₃CO—DWLKAFYDKVAEKLK—EAF—NH₂

Ac-15A-NH₂: CH₃CO—KAFYDKVAEKLKEAF—NH₂

Ac-12A-NH₂: CH₃CO—YDKVAEKLKEAF—NH₂

Ac-9A-NH₂ : CH₃CO—VAEKLKEAF—NH₂

going from one peptide to the other (top→bottom) 3 amino acids were removed from the N-terminus. The inhibitory effect of the four peptides listed above were compared at 120 μg peptide/ml. The lipid uptake by BBMVs was measured in the presence of 120 μg peptide/ml each as described below.

Donor and acceptor particles dispersed in Tris/NaCl buffer were centrifuged in a Beckman airfuge at 115000 g is for 2 min at 4° C. The dispersion of the acceptor yielded a pellet which was resuspended in Tris/NaCl buffer. Varying amounts of inhibitor dissolved in the same buffer were added to the acceptor dispersion and at time zero the dispersiorn of acceptor with or without inhibitor was mixed with the top 80% of the supernatant obtained by centrifugation of the donor dispersion. The final concentration of the donor was 0.05 mg total lipid/ml and that of the acceptor was 5 mg protein/ml. The resulting dispersion was incubated at 23° C. and after timed intervals the sterol uptake was stopped by dilution of the incubation medium with 2 volumes of Tris/NaCl buffer. BBMV were separated from the donor by centrifugation in the Beckman airfuge at 115000 g for 2 min at 4° C. The radioactivities in the supernatant containing the donor and in the pellet containing the BBMV (acceptor) were determined in a Beckman LS 7500 scintillation counter.

The results are expressed as % of inhibition and are summarised in Table 8 below:

TABLE 8

| | | Inhibition of Cholesteryloleate |
|---|---|---|
| No. | Peptide | Uptake In. % |
| 1 | Ac-18A-NH₂ | 100 |
| 2 | Ac-15A-NH₂ | 85 |
| 3 | Ac-12A-NH₂ | 20 |
| 4 | Ac-9A-NH₂ | 0 |

Useful inhibitory effect is seen with Peptides Nos. 1 and 2.

1. The use of a molecule comprising one or more amphipathic regions in the preparation of a medicament for inhibiting the uptake of cholesterol or other lipids from the gut.

2. The use of a molecule comprising one or more amphipathic regions in the preparation of a medicament for enteral administration for treating or preventing hyperlipidaemia, especially hypercholesterolaemia, and/or obesity.

3. The use as claimed in claim 1 or 2, wherein the molecule is a peptide or protein having one or more amphipathic α-helices.

4. The use as claimed in claim 3, wherein the molecule is an apoprotein.

5. The use as claimed in claim 4, wherein the apoprotein is an apoprotein A.

6. The use as claimed in claim 5, wherein the apoprotein is apoprotein A-1.

7. The use as claimed in claim 5, wherein the apoprotein is apoprotein A-2.

8. The use as claimed in claim 5, wherein the apoprotein is apoprotein A-4.

9. The use as claimed in claim 4, wherein the apoprotein is an apoprotein B.

10. The use as claimed in claim 9, wherein the apoprotein is apoprotein B-48.

11. The use as claimed in claim 9, wherein the apoprotein is apoprotein B-100.

12. The use as claimed in claim 4, wherein the apoprotein is an apoprotein C.

13. The use as claimed in claim 12, wherein the apoprotein is apoprotein C-1.

14. The use as claimed in claim 12, wherein the apoprotein is apoprotein C-2.

15. The use as claimed in claim 12, wherein the apoprotein is apoprotein C-3.

16. The use as claimed in claim 4, wherein the apoprotein is apoprotein D.

17. The use as claimed in claim 4, wherein the apoprotein is apoprotein E.

18. The use as claimed in claim 3, wherein the molecule is a variant of a natural apoprotein.

19. The use as claimed in claim 3, wherein the peptide or protein comprises one or more, but fewer than eight, amphipathic helices.

20. The use as claimed in claim 3, 18 or 19, wherein the molecule comprises one or more peptides of the sequence:
A₁—B₁—B₂—C₁—D—B₃—B₄—A₂—C₂—B₅—B₆—A₃—C₃—B₇—C₄—A₄—B₈—B₉ (I)
wherein
each of A₁, A₂, A₃ and A₄ independently represents aspartic acid or glutamic acid, or homologues or analogues thereof;
each of B₁, B₂, B₃, B₄, B₅, B₆, B₇, B₈ and B₉ independently represents tryptophan, phenylalanine, alanine, leucine, tyrosine, isoleucine, valine or α-naphthylalanine, or homologues or analogues thereof;
each of C₁, C₂, C₃ and C₄ independently represents lysine or arginine; and
D represents serine, threonine, alanine, glycine or histidine, or homologues or analogues thereof;
and wherein residues A₄, B₈ and B₉ are optional.

21. The use as claimed in claim 20, wherein the molecule is Ac-18A-NH₂ or Ac-15A-NH₂ or the corresponding unblocked or alternatively block form of either.

22. The use as claimed in any one of claims 4 to 17, wherein the apoprotein has been isolated from natural sources.

23. The use as claimed in claim 22, wherein the apoprotein has been purified to protein homogeneity (in the sense that no other proteins are present).

24. The use as claimed in claim 22, wherein the apoprotein has been purified to total homogeneity (in the sense that no significant amount of other molecules are present).

25. The use as claimed in any one of claims 3 to 21, wherein the peptide or protein has been prepared by recombinant DNA technology or peptide synthesis.

26. The use as claimed in claim 4, wherein the apoprotein(s) is or are in the form of lipoprotein.

27. The use as claimed in claim 26, wherein the lipoprotein comprises chylomicrons.

28. The use as claimed in claim 26, wherein the lipoprotein comprises chylomicron remnants.

29. The use as; claimed in claim 26, wherein the lipoprotein comprises VLDL.

30. The use as claimed in claim 26, wherein the lipoprotein comprises IDL.

31. The use as claimed in claim 26, wherein the lipoprotein comprises LDL.

32. The use as claimed in claim 26, wherein the lipoprotein comprises HDL.

33. The use as claimed in claim 1 or 2, wherein the molecule comprises amino acid residues at least some of which are linked by non-peptide bonds.

34. The use as claimed in claim 3, wherein at least one amino acid is a D-amino acid.

35. The use as claimed in claim 1 or 2, wherein the molecule is or comprises a synthetic peptidomimetic.

36. The use as claimed in claim 1 or 2, wherein the molecule comprises sugar and/or lipid moieties.

37. A formulation comprising one or more molecules as defined in any one of claims 1 to 36 and a pharmaceutically or veterinarily acceptable carrier, the formulation being adapted for enteral administration.

38. A formulation as claimed in claim 37, which is in unit dose form.

39. A formulation as claimed in claim 37 or 38, which is adapted for oral administration.

40. A formulation as claimed in claim 37, 38 or 39, which is in solid form.

41. A formulation as claimed in claim 39 or 40, which is formulated for enteric resistance.

42. A formulation as claimed in claim 37, which is formulated for rectal administration.

43. A formulation as claimed in any one of claims 37 to 42, comprising a cholesterol biosynthesis inhibitor.

44. A formulation as claimed in claim 43, wherein the cholesterol biosynthesis inhibitor is an inhibitor of 3-hydroxy-3-methylglutaryl coenzyme A (HMG—CoA) reductase.

45. A formulation as claimed in claim 44, wherein the HMG—CoA reductase inhibitor is a statin.

46. A formulation as claimed in claim 45, wherein the statin is simvastatin.

47. A product comprising a molecule as defined in any one of claims 1 to 36 and a cholesterol biosynthesis inhibitor for combined, separate or sequential administration in hypercholesterolaemia, or other hyperlipidaemia, prophylaxis or therapy and/or in the prophylaxis or treatment of obesity.

48. A product as claimed in claim 47, wherein the cholesterol biosynthesis inhibitor is an inhibitor of 3-hydroxy-3-methylglutaryl coenzyme A (HMG—CoA) reductase.

49. A product as claimed in claim 48, wherein the HMG—CoA reductase inhibitor is a statin.

50. A product as claimed in claim 49, wherein the stalin is simvastatin.

51. A method of treating or preventing hyperlipidaemia or hypercholesterolaemia, the method comprising enterally administering to a patient or subject an effective amount of a molecule comprising one or more amphipathic regions.

52. A method of treating or preventing obesity the method comprising enterally administering to a patient or subject an effective amount of a molecule comprising one or more amphipathic regions.

53. A method of treating or preventing atherosclerosis, the method comprising enterally administering to a patient or subject an effective amount of an apoprotein.
